(19) United States
(12) Patent Application Publication
(10) Pub. No.: US 2024/0018459 A1
(43) Pub. Date: Jan. 18, 2024

(54) ANALYZING APPARATUS, ANALYZING SYSTEM, ANALYZING METHOD, AND COMPUTER-READABLE NON-TRANSITORY STORAGE MEDIUM

(71) Applicant: HITACHI HIGH-TECH SOLUTIONS CORPORATION, Tokyo (JP)

(72) Inventors: Junya KOSAKA, Tokyo (JP); Toshimitsu SHIRAISHI, Tokyo (JP)

(21) Appl. No.: 18/373,452

(22) Filed: Sep. 27, 2023

Related U.S. Application Data
(63) Continuation of application No. PCT/JP2022/007242, filed on Feb. 22, 2022.

(30) Foreign Application Priority Data
Mar. 30, 2021 (JP) 2021-057121

(51) Int. Cl. C12M 1/34; C12M 1/00; C12M 1/36

(52) U.S. Cl. CPC C12M 41/46; C12M 41/06; C12M 41/48

(57) ABSTRACT

The analyzing apparatus includes a unit to acquire a plurality of first images captured with first visible light in time-series during a culturing step, the plurality of first images acquired by capturing an interior of the sampling container having undergone a process of sampling microorganisms in a monitored environment, a first specifying unit to detect occurrence of a colony of the microorganisms or an existence of the colony having occurred already in the time-series from time-series variations of the plurality of the first images, and to specify a shape of the colony with its occurrence or existence being detected, a second specifying unit to specify the shape of the colony of the microorganisms from a second image acquired by capturing the interior of the sampling container with second visible light, and a unit to display a state of the colony of the microorganisms.

### CROSS REFERENCE TO THE RELATED APPLICATION

This application is a continuation application of International Application PCT/JP2022/007242 filed on Feb. 22, 2022 and designated the U.S., the International Application PCT/JP2022/007242 is based upon and claims the benefit of priority of the prior Japanese Patent Application No. 2021-057121, filed on Mar. 30, 2021, the entire contents of both are incorporated herein by reference.

### BACKGROUND

### Technical Field

The invention pertains to an analyzing apparatus, an analyzing system, an analyzing method, and a computer-readable non-transitory storage medium storing a program.

### Description of the Related Art

In an environment for manufacturing products based on cell culture or bioproducts and other equivalents, or in an environment for researching and developing the above products, what is required involves securing safely manufacturing the products as the case may be. Such a case requires carrying out predetermined environmental monitoring. The environmental monitoring involves culturing and observing airborne bacteria, adherent bacteria and other equivalents existing within the environment where the products are manufactured, and ensuring that the environment is maintained to be equal to or lower than predetermined criteria against the bacteria. The environmental monitoring further involves eventually requiring a visual inspection for determination in person. Such being the case, technologies are proposed, which are related to a variety of image processing software to improve monitoring accuracy of cultured results.

### PRIOR ART DOCUMENTS

### Patent Documents

[Patent Document 1] Japanese Unexamined Patent Application Publication No. 1995-282220

### SUMMARY

However, an image easy to be determined by a person is not necessarily coincident with an image suited to processing by image processing software and other equivalents of a computer and other apparatuses. Consequently, a method of presenting an analysis result provided by the computer and other apparatuses has room for improvement in environment monitoring. Under such circumstances, it is an aspect of the present invention to provide an analyzing apparatus, an analyzing method, (an analyzing system), and a computer-readable non-transitory storage medium storing a program, which are capable of assisting a person in making a determination regarding a result of culturing microorganisms.

One aspect of the present invention is exemplified by an analyzing apparatus that analyzes a sampling container to sample microorganisms. The analyzing apparatus includes; a unit configured to acquire a plurality of first images, captured with first visible light in time-series during a culturing step, the plurality of first images acquired by capturing an interior of the sampling container having undergone a process of sampling microorganisms in a monitored environment; a first specifying unit configured to detect occurrence of the colony of the microorganisms or an existence of the colony occurring already in the time-series from time-series variations of the plurality of the first images, and to specify a shape of the colony with its occurrence or existence being detected; a second specifying unit configured to specify the shape of the colony of the microorganisms from a second image acquired by capturing the interior of the sampling container with second visible light; and a unit to display a state of the colony of the microorganisms, based on the shape specified by the first specifying unit and the shape specified by the second specifying unit.

The first specifying unit detects the occurrence of a colony of the microorganisms or the existence of the colony occurring already in the time-series from time-series variations of the plurality of the first images, and specifies the shape of the colony. Herein, the first image is captured with the first visible light suited to processing of the first image.

The second specifying unit specifies the shape of the colony of the microorganisms from second images, captured with second visible light different from the first visible light. The second image is captured with the second visible light suited to processing of the second image. The analyzing apparatus is therefore enabled to display, with respect to these two types of images, states of the colonies of the microorganisms, based on the result of detecting the occurrences of the colonies in the time-series in the first images and the result specified from the second images.

The analyzing apparatus is capable of effectively assisting a person in making a determination regarding a result of culturing microorganisms.

### BRIEF DESCRIPTION OF THE DRAWINGS

FIG. 1 IS A diagram illustrating a configuration of an analyzing apparatus;

FIG. 2 is a flowchart illustrating analysis processing procedures;

FIG. 3 is a flowchart illustrating the analysis processing procedures;

FIG. 4 is a flowchart illustrating the analysis processing procedures;

FIG. 5 is a flowchart illustrating details of a light quantity adjusting process;

FIG. 6 is a flowchart illustrating details of detecting an existing colony and a new colony;

FIG. 7 is a diagram illustrating time-series images of the colonies that occur and grow during a culturing step;

FIG. 8 is a diagram illustrating a process of aligning central positions of Schales PD;

FIG. 9 is a diagram illustrating a process of aligning rotation angles of the Schales PD;

FIG. 10 is a diagram illustrating a process of classifying the colonies in the images of this time into the existing colony and the new colonies;

FIG. 11 is a diagram illustrating an environment determination system according a second embodiment; and

FIG. 12 is a diagram illustrating an environment monitoring system according to a third embodiment.

### DESCRIPTION OF THE EMBODIMENTS

An analyzing apparatus and an analyzing method according to one embodiment will hereinafter be described with reference to the accompanying drawings.

### First Embodiment

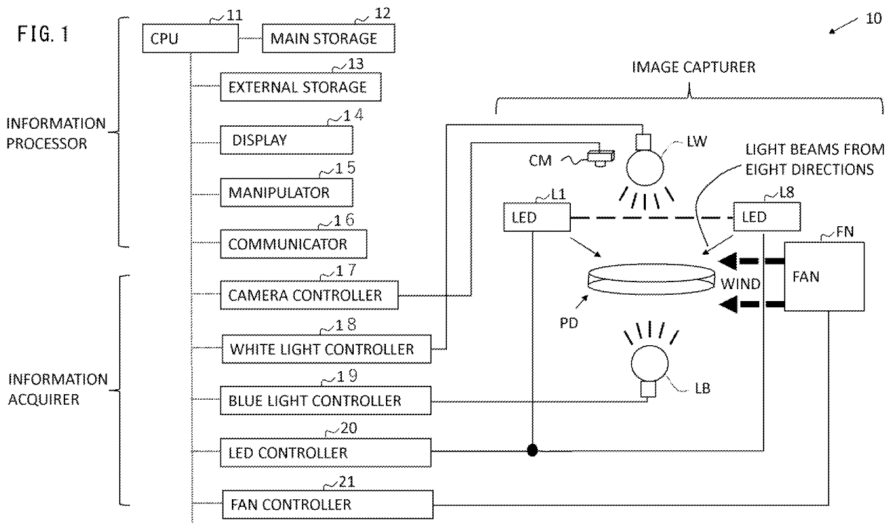

An analyzing apparatus 10 according to a first embodiment will be described with reference to FIGS. 1 through 10. The analyzing apparatus 10 analyzes a Schale PD that retains samples acquired in monitored environments exemplified by an environment for manufacturing products based on cell culture or bioproducts and other equivalents and a research and development environment. The monitored environments are also referred to as an aseptic room and a sterilized room. The Schale PD is also called a sampling container. The Schale PD is given another nomenclature called a Petri dish that is a transparent disc-shaped container. The Schale PD includes: a shallow cylindrical or dish-like main body with its upper portion being opened, which has a transparent bottom; and a transparent cover portion to cover and close the upper opening of the main body.

The Schale PD is placed, with its upper portion being opened by taking off the cover portion, in a predetermined position under the monitored environment, thus acquiring inspection target samples, which are microorganisms floating in the air or falling down as called, e.g., the airborne bacteria (which are also termed airborne microorganisms). The Schale PD acquires inspection target samples of the microorganisms called, e.g., the adherent bacteria, which adhere to objects placed in the monitored environment, human bodies or clothes of workers operating in the monitored environment. The microorganisms adhering to the human bodies or the clothes undergo a process of collecting the inspection target samples by getting a bottom surface of an inner wall of the Schale PD to contact inspection target portions.

The Schale PD having acquired the inspection target samples is placed in a culturing step to culture the microorganisms exemplified by the bacteria, molds and fungi. The analyzing apparatus 10 acquires images for every predetermined period from the Schale PD placed in the culturing step, and analyzes the images. The images are obtained by capturing the images of the microorganisms on the bottom surface of the interior of the Schale PD through, e.g., the transparent cover portion. The collected inspection target samples contain a predetermined or higher quantity of microorganisms, in which case the microorganisms undergo gradual development through the culturing step. The microorganisms, which develop and form clusters visible to human eyes, are referred to as colonies.

The analyzing apparatus 10 according to the first embodiment detects the colonies of the microorganisms from the images, in the Schale PD, of the microorganisms being cultured or after completing the cultivation in the culturing step, and assists a person (also called an operator or a user) in monitoring the environment.

(Configuration)

FIG. 1 is a diagram illustrating a configuration of the analyzing apparatus 10. As in FIG. 1, the analyzing apparatus 10 includes mainly an information processor, and information acquirer, and an image capturer. Though omitted in FIG. 1, the Schale PD is placed in a position enabling the image capturer to capture the image of the bottom surface of the inner wall of the Schale PD via the transparent cover portion. In that case, the analyzing apparatus 10 may include a placement plate on which the Schale PD is placed. The Schale PD may be retained in a position enabling the image capturer to capture the image of the bottom surface of the inner surface of the Schale PD via the transparent cover portion. In that case, the analyzing apparatus 10 may include a holder for holding the Schale PD.

The information processor of the analyzing apparatus 10 includes a Central Processing Unit (CPU 11), a main storage 12, and external devices connected (to the CPU 11) via an interface (I/F), and executes information processing based on programs. The CPU 11 is also called a processor. The external devices may be exemplified by an external storage 13, a display 14, a manipulator 15, and a communicator 16. The information processor may be a computer built in a housing of the analyzing apparatus 10, and may also be an ordinary personal computer as a separate computer from the housing of the analyzing apparatus 10.

The CPU 11 runs computer programs deployed in an executable manner on the main storage device 12, thereby providing functions of the analyzing apparatus 10. The CPU 11 is called a processor. The CPU 11 may take a multi-processor configuration without being limited to a single processor. The CPU 11 may be configured to include a Graphics Processing Unit (GPU), a Digital Signal Processor (DSP) and other equivalents. The CPU 11 may also be configured to cooperate with hardware circuits exemplified by a Field Programmable Gate Array (FPGA) and other equivalents. The main storage device 12 stores the computer programs that are run by the CPU 11, and data that are processed by the CPU 11.

The main storage device 12 is exemplified by a Dynamic Random Access Memory (DRAM), a Static Random Access Memory (SRAM), and a Read Only Memory (ROM). The external storage 13 is used as, e.g., a storage area auxiliary to the main storage device 12, and stores the computer programs to be run by the CPU 11 and data to be processed by the CPU 11. The external storage 13 is exemplified by a hard disc drive and a Solid State Drive (SSD). The analyzing apparatus 10 may further be provided with a drive device for detachable storage mediums. The detachable storage mediums are exemplified by a Blu-ray disc, a Digital Versatile Disk (DVD), a Compact Disc (CD), a flash memory card, and a Cloud storage. Herein, the Cloud storage is an external storage system connected by a network.

The display 14 is exemplified by a liquid crystal display, and an electroluminescence panel. The manipulator 15 is exemplified by a keyboard, and a pointing device. In the first embodiment, the pointing device is exemplified by a mouse, a touch pad, and a touch panel. The communicator 16 receives and transfers the data from and to other devices on the network.

The information acquirer of the analyzing apparatus 10 is connected to the information processor via a predetermined interface. The information acquirer controls the image capturer, adjusts an image capturing environment, acquires the images from the image capturer, and hands over the images to the information processor. The information acquirer includes a camera controller 17, a white light controller 18, a blue light controller 19, an LED controller 20, and a fan controller 21. The image capturer sets image capturing conditions by projecting illumination light on the Schale PD and cleaning off contaminants adhered to the surface of the Schale PD, and thus captures the images of the Schale PD. The image capturer includes a camera CM, a white light LW, a blue light LB, LEDs L1-L8, and a fan FN.

The camera controller 17 acquires the images from the camera CM, and retains the acquired images on an image memory. The camera controller 17 hands over the images on the image memory to the CPU 11. The camera CM is exemplified by a Charge Coupled Device (CCD) camera, and a Complementary Metal Oxide Semiconductor (CMOS) camera. The camera CM hands over the image data to the camera controller 17 at a predetermined period, and gets the image memory to store the image data.

The white light controller 18, in response to a command from the CPU 11, controls switching the white light LW On and Off, and controls a light quantity of the white light irradiated from the white light LW. The white light LW irradiates the light emitted from a blue Light Emitting Diode (LED) over a fluorophore, thus emitting the white light. The white light LW includes a semiconductor switch, and is switched ON by a control signal coming from the white light controller 18 to thereby emit the white light. The white light LW includes, e.g., a dimmer circuit. The dimmer circuit includes, e.g., a rectifier circuit, a DC (Direct Current)-DC converter, and controls a quantity of electric current flowing through the blue LED by controlling a pulse width (duty ratio) of the DC-DC converter, and adjusts the light quantity emitted from the white light LW. The white light controller 18 outputs, to the white light LW, a signal of an On-Off command of the semiconductor switch and a signal for specifying the pulse width (duty ratio) of the DC-DC converter. Note that the white light controller 18 may also be configured to include the dimmer circuit and to supply electricity while controlling a value of the electric current to the white light LW.

The blue light controller 19, in response to a command from the CPU 11, controls switching the blue light LB On and Off, and controls a light quantity of the blue light irradiated from the blue light LB. The blue light LB includes, e.g., the blue LEDs or an array of the blue LEDs. The blue light LB may have the fluorophore that emits the light having a wavelength of blue. The blue light LB irradiates the blue light from the underside of the Schale PD. The camera CM captures the image of the bottom surface of the Schale PD over which the blue light LB projects the blue illumination light from under through the upper transparent cover portion. In simpler words, the blue light LB irradiates the blue transmitted light over the Schale PD.

The blue light LB includes the same semiconductor switch and the same dimmer circuit as those of the white light LW. The blue light controller 19 outputs, to the blue light LB, a signal for specifying an On-Off command of the semiconductor switch and a signal for specifying the pulse width (duty ratio) of the DC-DC converter. Note that the blue light controller 19 may also be configured to include the dimmer circuit and to supply the electricity while controlling the value of the electric current to the blue light LB. The camera CM captures a first image of the Schale PD by using the transmitted light from the blue light LB, and a second image by using reflected light from the white light LW. The camera CM is therefore one example of an image capturing unit to capture the first image and the second image. Note that the image captured by using the transmitted light from the blue light LB is defined as an image suited to processing based on the computer programs exemplified by image processing software. This is because a simple color is suited to the image processing software. The inventors of the analyzing apparatus 10 empirically recognize that the image captured by using the blue light LB is higher in accuracy of detecting the colonies than in the case of using the light of other colors.

The LED controller 20 performs On-Off control of the eight LEDs L1-L8. Though omitted in FIG. 1, the LEDs L1-L8 are arrayed to circularly illuminate the vicinity of center of the Schale PD at an angle of 45 degrees from above the Schale PD. The LEDs L1-L8 emit white light beams each having a directivity in predetermined directions. Accordingly, the LEDs L1-L8 irradiate the light beams having the directivities toward the center of the bottom surface of the inner wall from the eight directions above the Schale PD through the cover portion. It does not, however, mean that a number of the LEDs L is limited to “8”. For instance, the image capturer may be provided with the four LEDs in the four directions. The eight LEDs L1-L8 operate to three-dimensionally capture the images of the colonies of the microorganisms within the Schale PD by using the white light beams having the directivities from the different directions. With these operations, the analyzing apparatus 10 assists the user in making it distinguishable whether shadow areas in the captured image of the Schale PD are the three-dimensionally structured colonies, or flat contaminants or tarnishes. The white light beams from the LEDs L1-L8 are emitted from the blue LED and the fluorophore in the same way as the white light LW.

The fan controller 21 conducts On-Off control of the fan FN. Upon switched ON, the fan FN applies a predetermined amount of air blow (indicated by dotted bold arrows) to the Schale PD by rotating blades. With the action of the fan FN, the analyzing apparatus 10 cleans off foreign matters, i.e., contaminants adhered to the surface of the Schale PD placed or retained under the camera CM. The CPU 11, before the camera CM captures the image, commands the fan controller 21 to switch ON the fan FN or repeat the On-Off operations a predetermined number of times, thereby cleaning off the foreign matters on the surface of the Schale PD. A desirable fan FN is capable of applying the air blow to both of the surface of the Schale PD, especially an upper surface of the cover portion and the bottom surface of the main body. The fan FN is one example of an air blowing unit. In other words, the fan FN applies the air blow to the surface of the sampling container placed in a state enabling the camera CM as the image capturing unit to capture the image.

Note that a carrying mechanism, though not illustrated in FIG. 1, may also be provided, which carries the Schale PD placed in the culturing step to the placement plate or the holder in a position enabling the image capturer of the analyzing apparatus 10 to capture the image.

(Processing)

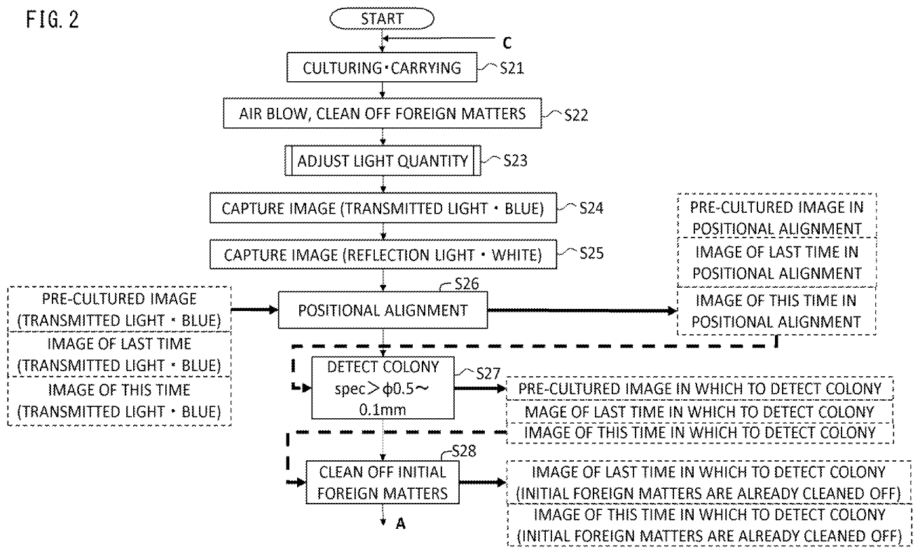

A processing procedure of an analyzing method in the analyzing apparatus 10 will be illustrated with reference to FIGS. 2 through 6. The processes are executed by the CPU 11 in accordance with the computer programs deployed in the executable manner on the main storage 12. However, the following description will be made on the assumption that the analyzing apparatus 10 executes the processes. FIGS. 2 through 4 among FIGS. 2 through 6 are flowcharts illustrating analysis processing procedures. The processes illustrated in FIGS. 2 through 4 are executed, with a first process being a process before the Schale PD enters the culturing step, a predetermined number of times at a predetermined timing in the culturing step.

In this process, to begin with, the analyzing apparatus 10 carries the Schale PD placed in the culturing step, and sets a status to enable the image capturer to capture the image of the bottom surface of the inner wall of the Schale PD (S21). When the analyzing apparatus 10 does not include that carrying mechanism, however, the Schale PD is set in the status enabling the image of the bottom surface of the inner wall of the Schale PD to be captured through an intermediary of the operator. In this case, the analyzing apparatus 10 recognizes that the Schale PD completes being placed and is set in the analysis-enabled status by the input of the operator via the manipulator 15. The analyzing apparatus 10 may recognize that the Schale PD completes being placed and is set in the analysis-enabled status from signals of a switch provided on the placement plate on which the Schale PD is placed and a switch provided in the holder to hold the Schale PD.

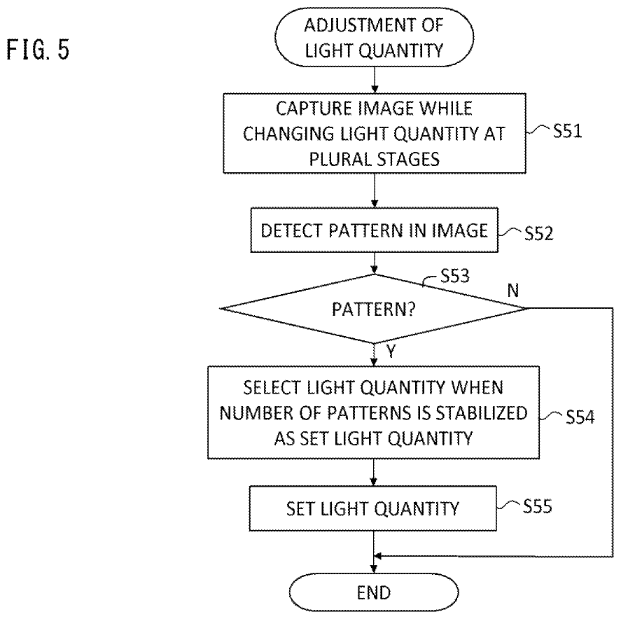

Next, the analyzing apparatus 10 causes the fan controller 21 to operate the fan FN to apply the air blow to the Schale PD, thereby cleaning off the foreign matters on the surface of the Schale PD (S22). Subsequently, the analyzing apparatus 10 adjusts the light quantities of the white light LW and the blue light LB (S23). A detailed description of the process of adjusting the light quantities will be made separately referring to FIG. 5.

The analyzing apparatus 10 causes the camera CM to capture the image of the Schale PD in such a status that the blue light LB irradiates the transmitted light from under the Schale PD (S24). The light quantities are adjusted in S23. The analyzing apparatus 10 iterates the processes in FIGS. 2 through 4 in time-series a plural number of times as the culturing step advances. The process in S24 is therefore one example of acquiring a plurality of first images, within the Schale PD defined as the sampling container, captured with first visible blue light in the time-series in the culturing step. The CPU 11 of the analyzing apparatus 10 acquires the images captured in the process of S24, executed the plural number of times in the time-series, through the camera controller 17. Accordingly, the CPU 11 may also be said to be a unit configured to acquire the plurality of first images.

Subsequently, the analyzing apparatus 10 causes the camera CM to capture the image of the Schale PD in such a status that the white light LW irradiates the light from above the Schale PD (S25). The camera CM captures the image with the reflected white light irradiated from the white light LW. The light quantity of the white light irradiated from the white light LW is adjusted in S23.

The analyzing apparatus 10 executes following processes in S26 through S42 on the images captured in the status of irradiating the transmitted light from the blue light LB. To be specific, the analyzing apparatus 10 conducts positional alignment of the image (previous image) already captured in the process of S24 in the last culturing step with the image (the image of this time) captured with the blue light this time (S26). Note that the previous image includes the image before being cultured. For example, the main storage device 12 stores the pre-cultured images captured in the first process before the Schale PD enters the culturing step, and the images captured up to last time in the subsequent processes. The processes of FIGS. 2 through 4 involve executing a process of detecting occurrence of the colonies or a process of specifying the detected colonies by comparing the already-captured images with the images of this time. As pre-processing of the image comparisons performed as such, the analyzing apparatus 10 carries out the positional alignment of the images with each other.

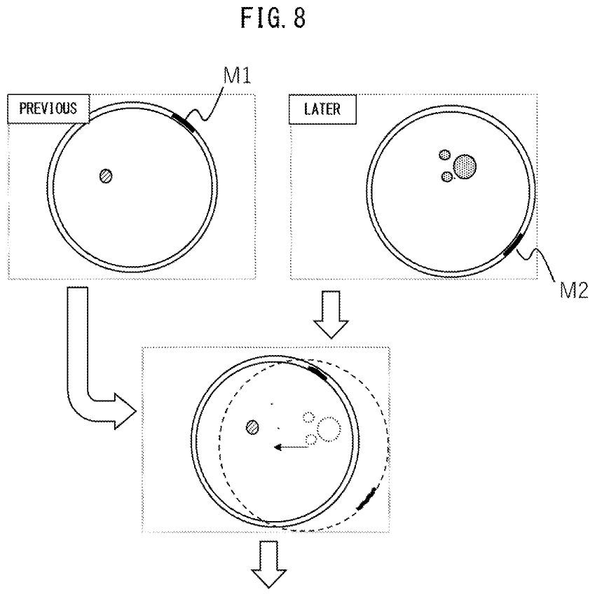

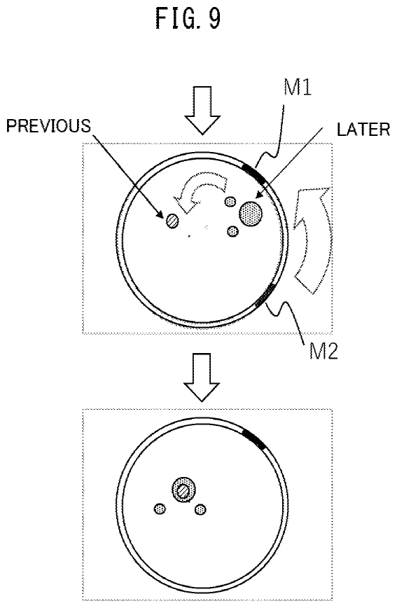

As illustrated FIGS. 8 and 9, edge portions of the upper surfaces of the Schales PD are formed with a mark M1 and a mark M2. The positional alignment includes a process of aligning central positions of Schales PD with each other, and a process of rotating one of the two images so that the mark M1, M2 on the edge portion of one image is overlapped with another mark M1, M2 on another image.

The analyzing apparatus 10 executes a process of detecting the colonies from the positionally-aligned images of the last time and from the images of this time (S27). This process is a process of detecting the shadow areas, equal to larger than a predetermined dimension, in the images of this time. The predetermined dimensions is saved as, e.g., a system parameter in the main storage 12. Note that the colonies in the images of the last time are already detected in the previous process, and hence the analyzing apparatus 10 may simply read detection results of the previous images from the main storage 12.

The analyzing apparatus 10 cleans off initial foreign matters from the images of this time (S28). The analyzing apparatus 10 saves, in the main storage 12, the pre-cultured images captured in the first process before the Schale PD enters the culturing step. The shadow areas in the pre-cultured images are shadow areas of the foreign matters and noises other than the colonies. The images of this time contain the same shadow areas as those in the pre-cultured images, in which case the analyzing apparatus 10 therefore removes these shadow areas (S28). The removal of the same shadow areas is attainable by subtracting the shadow areas in the pre-cultured images from the shadow areas in the images of this time. The process of removing the same shadow areas is provided by the image processing software installed in the CPU 11. The analyzing apparatus 10 saves, in the main storage 12, the images, from which the initial foreign matters are already cleaned off, of this time (the colony-detected images of this time in FIG. 2). Note that the images, from which the initial foreign matters are already cleaned off, of the last time (the colony-detected image of the last time in FIG. 2) are already saved in the main storage 12.

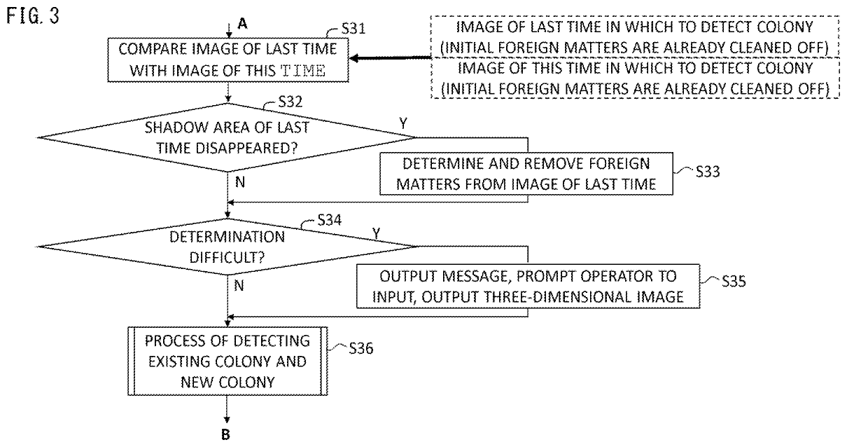

The processes will next be described by referring to FIG. 3. An arrow A in FIG. 2 indicates that the processing continues to the arrow A in FIG. 3. The analyzing apparatus 10 compares the two images from which the initial foreign matters are cleaned off, i.e., compares the image of the last time with the image of this time (S31). When the shadow area existing in the image of the last time disappears in the image of this time (Y in S32), the analyzing apparatus 10 recognizes that the disappeared shadow area is the foreign matter, and deletes the colony (taking a polygonal shape) of the concerned area of the detected result in the image of the last time (S33). The analyzing apparatus 10 subtracts a number of the colonies corresponding to the disappeared shadow areas from the number of the colonies detected up to the last time. The process in S33 is one example of a process of excluding the shadow area existing in the previous image as the shadow area other than the colonies of the microorganisms when the shadow area existing in the previous image does not exist in the later image among the previous and later images in the time-series. The determination in S32 is one example of a process of distinguishing between the shadow areas of the colonies of the microorganisms and the shadow areas other than the colonies of the microorganisms. Accordingly, the CPU 11 of the analyzing apparatus 10 executes the determination in S32 by way of one example of a distinguishing unit.

However, it is difficult to compare the image of the last time with the image of this time (Y in S34), in which case the analyzing apparatus 10 prompts the operator to input a result of the distinguishment by outputting a message to the display 14 (S35). At that time, the analyzing apparatus 10 may output, to the display 14, the three-dimensional images captured by irradiating the light beams of the LEDs L1-L8 from the eight directions. The difficult case of the determination in S34 may be said to be a difficult case of the distinguishment by the distinguishing unit. The three-dimensional images captured by irradiating the light beams from the eight directions are one example of images captured in a status of illuminating the Schale PD serving as the sampling container with illumination light beams from a plurality of directions. Note that the three-dimensional images may also be images captured by irradiating the light beams from, e.g., four directions other than the eight directions.

Herein, the difficult case of the determination is a case that a new shadow area appears in the image in the last colony detection process after finishing the culturing step, and a case of being unable to check whether the newly-appeared shadow area disappears next time. The analyzing apparatus 10 retains, e.g., information indicating whether the process of this time is the last colony detection process, or information indicating a number of executions of the colony detection process in the culturing step. The analyzing apparatus 10 is therefore enabled to recognize whether the image of this time is the image in the last colony detection process after finishing the culturing step. The difficult case of the determination is exemplified by a difficult case of determining whether the shadow area disappears because the image of this time contains a shadow area that is partially overlapped with the shadow area in the image of the last time. In such a case, the operator inputs distinguishment result, thereby determining whether the shadow area is the foreign matter. At that time, the analyzing apparatus 10 assists the determination of the operator with the aid of the three-dimensional images captured with the light beams irradiated by the LEDs L1-L8 from the eight directions. The process in S35 is one example of a process of presenting information to prompt an intermediary of the operator. The CPU 11 of the analyzing apparatus 10 executes the process om S35 by way of one example of a providing unit.

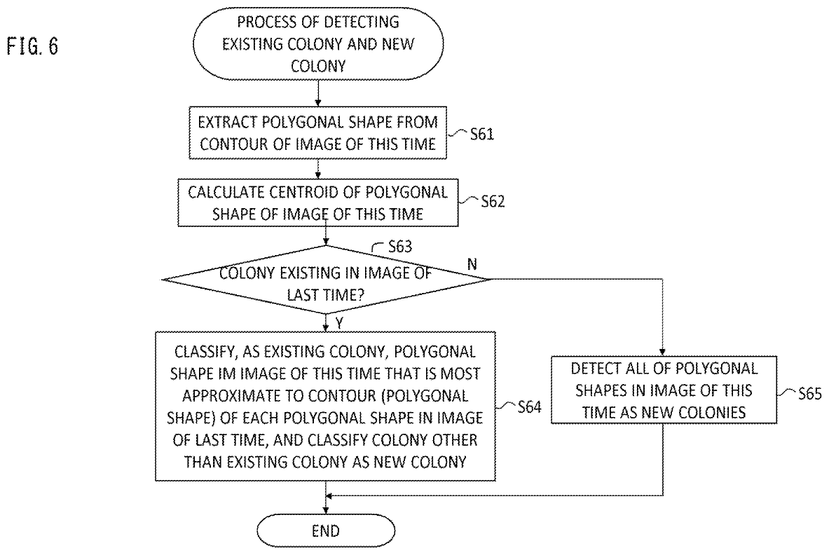

The analyzing apparatus 10 executes a process of detecting an existing colony and a new colony (S36). The process of detecting the existing colony and the new colony will be separately described with reference to FIG. 6. Herein, the process is executed on the images captured at respective stages of the culturing step and saved in the time-series (the images captured in the status of irradiating the transmitted light from the blue light LB. To be specific, the analyzing apparatus 10 specifies the newly-occurred colony and the existing colony by comparing the respective previous images with the images saved in the time-series, and extracts respective contours and centroids thereof.

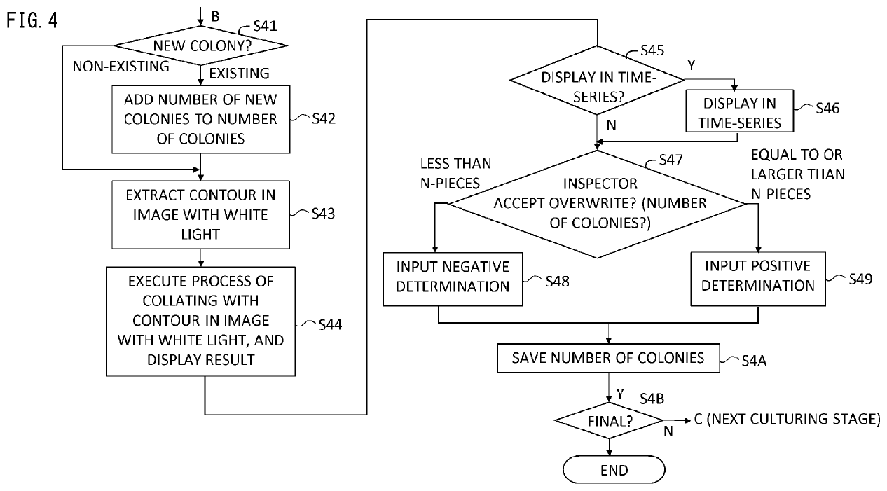

The processes will next be described with reference to FIG. 4. An arrow B in FIG. 3 indicates that the processing continues to the arrow B in FIG. 4. The analyzing apparatus 10 determines whether the new colony occurs in the image of this time as a result of the process in S36 (S41). When the new colonies occur in the image of this time, a number of the new colonies is added to the number of the colonies.

The analyzing apparatus 10 extracts the contour from the image captured with the reflected light of the white light irradiated from the white light LW (S43). The process in S43 may be said to be a process of specifying a shape of the colony of the microorganisms from the second image, captured with the white light as second visible light, of the interior of the Schale PD defined as the sampling container. This process is also executed at the respective stages of the culturing step. The process in S43 is therefore one example of the process of specifying the shape of the colony of the microorganisms from the second image, captured with the white light as the second visible light, of the interior of the Schale PD defined as the sampling container. Note that the CPU 11 of the analyzing apparatus 10, which executes the process of detecting the existing colony and the new colony, is called a first specifying unit, and the processes in FIG. 6 are generically referred to as a first step of detecting occurrence of the colony of the microorganisms or an existence of the colony occurring already in the first embodiment. This first step is the process of detecting the colony from the image captured with the blue transmitted light. On the other hand, the CPU 11 of the analyzing apparatus 10, which executes the process in S43 is called a second specifying unit, and the process in S43 is referred to as a second step of specifying the shape of the colony of the microorganisms.

The analyzing apparatus 10 executes a process of collating the contour of the existing colony with the contour of the new colony, which are acquired in the process of detecting the existing colony and the new colony in S36, and displays a processing result on the display 14 (S44). Herein, the analyzing apparatus 10 determines the colony contour from the contours extracted from the images captured previously and lately in the time-series with the blue transmitted light, and from the contours extracted from the images captured with the reflected light of the white light in the process of S43. For instance, the analyzing apparatus 10 overlaps the colony contour acquired in the detection process of S36 with the contour extracted in the process of S43, and displays the overlapped colony contour. However, the analyzing apparatus 10 may, when having a discordance between the contour acquired in the detection process of S36 and the contour acquired in the process of S43, prioritize the contour acquired in the detection process of S36 with respect to the discordant area. A reason why so is that the contour is acquired from the series of images captured at the respective stages of the culturing step in the detection process of S36, and hence the detection result is high in reliability. The detection process in S36 involves using the images captured in the status of performing the irradiation of the transmitted light from the blue light LB, and therefore the result of the image processing is empirically high in reliability. For example, the analyzing apparatus 10 may simply not adopt, as the contour, the area apart at a distance equal to or larger than a predetermined limit from the contour given in the detection process of S36 among the contours extracted in the process of S43.

Note that the contour looks as one contour in the images captured with the reflected light of the white light, and, however, the plural colonies are actually overlapped with each other as the case may be. Therefore, the analyzing apparatus 10 provides displaying by adding the contour of the colonies onto the images captured with the reflected light of the white light, and retains the counted value in the process of S42 as the counted number of colonies. Accordingly, the analyzing apparatus 10 provides displaying by overlapping, the colony (polygonal shape) given in the time-series processing of the image of the blue light and the colony having the confirmed coincidence of the edges of the shadow areas on the image of the white light, with the image, captured with the reflected light of the white light, that the operator looks at. The analyzing apparatus 10 provides the operator with the number of the colonies acquired so far in the time-series process. Herein, the items of information exemplified by the colony contour, the image captured with the white light and the number of the colonies, may be said to be information representing a state of the colony of the microorganisms, based on the shape specified by the first specifying unit (FIG. 6) and the shape specified by the second specifying unit (S43). The CPU 11 of the analyzing apparatus 10 executes the process in S44 as a unit to display the state of the colony of the microorganisms.

When the operator requests time-series display through manipulation of the manipulator 15 of the analyzing apparatus 10 (Y in S45), the analyzing apparatus 10 carries out the time-series display. In the time-series display, the analyzing apparatus 10 displays in the time-series the already-extracted colony contours at the respective stages of the culturing step, or the images to be processed at the respective stages (S46). At that time, the operator is able to display, on the display 14, the colony contours at the respective stages or the images to be processed at the respective stages in a way that freely magnifies and minifies the contours or the images through the manipulation of the manipulator 15. The process in S46 is one example of a process of displaying the state of the colony in the time-series. The CPU 11 of the analyzing apparatus 10 executes the process in S46 by way of one example of a unit to display the state of the colony in the time-series.

Based on the items of information exemplified by the colony contours, the images and the number of the counted colonies, which are displayed in the processes of S44 through S46, the operator (also termed an inspector) determines whether the colonies exist (S47). To be specific, the number of the detected colonies is less than a reference value (n-pieces), e.g., is “0”, in which case the operator inputs “negativity determined” (S48). Whereas when the number of the detected colonies is equal to or larger than the reference value (n-pieces), the operator inputs “positivity determined” (S49). The processes in S48 and S49 may be said to be a process of accepting confirmation of the user with respect to whether the microorganisms exist, judging from the displayed state of the colonies. The CPU 11 of the analyzing apparatus 10 executes the processes in S48 and S49, serving as a unit to accept the confirmation of the user. The analyzing apparatus 10 overwrite-saves the number of the detected colonies and other equivalent items together with results of the determinations of the positivity and the negativity (S4A).

The analyzing apparatus 10 determines whether the process of this time is a process at a final stage after finishing the culturing step (S4B). When the process of this time is the process at the final stage, the analyzing apparatus 10 finishes processing on the Schale PD. Whereas when the process of this time is not the process at the final stage, the analyzing apparatus 10 moves this Schale PD back to the next stage of the culturing step (S21). After finishing the next stage, the analyzing apparatus 10 executes the processes from S22 onward. An arrow C in FIG. 4 indicates the process continuation as indicated by the arrow C in FIG. 2.

FIG. 5 illustrates details of the light quantity adjusting process (S23 in FIG. 2). A reason why to adjust the light quantity herein is that a proper light quantity may differ depending on the cultured state of the microorganisms in the Schale PD. For instance, there is a small area occupied by the colony on the bottom surface of the inner wall of the Schale PD, in which case a large light quantity results in excessive brightness of the image captured by the camera CM of the image capturer. In that case, even when the colony exists in the Schale PD, such a case might occur that the shadow area of the colony is not left in the captured image.

While on the other hand, there is a large area occupied by the colony on the bottom surface of the inner wall of the Schale PD, in which case a small light quantity results in excessive darkness of the image captured by the camera CM of the image capturer. In that case, even when the multiple colonies exist in the Schale PD, such a case might occur that the shadow areas of the colonies are not clearly distinguishable in the captured image. Such being the case, the analyzing apparatus 10 counts the number of the colonies of each of the plural images captured while changing the light quantity, and searches for a stable state exhibiting a less variation in the number of the colonies. The light quantity in this stable state is set as a desirable light quantity. The following processes are carried out for both of the blue light LB and the white light LW. The processes for the blue light LB are herein exemplified, however, the same processes are applied to the white light LW. To be specific, the analyzing apparatus 10 detects the number of the colonies from the images with the light quantity, being stepwise changed, of one of the blue light LB as the first visible light and the white light LW as the second visible light. The description will be made as below by exemplifying the blue light LB.

In this process, the analyzing apparatus 10 captures the image of the Schale PD while changing the light quantity of the blue light LB at a plurality of stages (S51). The images to be captured herein are one example of the plurality of images captured while changing the light quantity stepwise. The analyzing apparatus 10 counts a number of patterns in the respective images (S52). The number of the patterns is counted by transforming the shadow areas in the images into polygonal shapes (IDs and a train of vertexes) and counting a number of polygonal shapes (a number of IDs).

The analyzing apparatus 10 confirms that the pattern exists in any one of the images, i.e., the number of the patterns is not “0” (S53). Namely, this is because the number of the patterns does not vary even by changing the light quantity at the plurality stages when none of the colonies exist in the Schale PD.

When the number of the patterns is not “0” (Y in S53), the analyzing apparatus 10 detects the light quantity in such a status that the variation in the number of the patterns is equal to or smaller than a predetermined threshold value, e.g., in a status of having no variation in the number of the patterns, and determines the detected light quantity as a proper light quantity (S54). Herein, the status of having no variation in the number of the patterns is one example of such a stage that a variation in the number of shadow areas among the plural images captured while changing the light quantity stepwise fulfills a predetermined condition. The analyzing apparatus 10 sets the light quantity of the blue light LB on the basis of the proper light quantity determined in the process of S54 (S55). The process in S55 is one example of a process of setting to the light quantity when capturing the image.

The blue light LB is one example of the first visible light. The white light LW is one example of the second visible light. Accordingly, the CPU 11 of the analyzing apparatus 10 executes the processes in FIG. 5 by way of one example of a unit to set to the light quantity.

FIG. 6 is a flowchart illustrating details of the process of detecting the existing colony and the new colony (S36 in FIG. 3). In this process, the analyzing apparatus 10 extracts the polygonal shape from the contours of the images of this time previously and lately in the time-series (S61). The analyzing apparatus 10 calculates the centroid of the polygonal shape of the image of this time (S62). The analyzing apparatus 10 determines whether the colony is already detected in the image of the last time (S63). When the colony is already detected in the image of the last time (Y in S63), the analyzing apparatus 10 detects occurrence of a new colony by associating the colony already detected in the image of the last time with the colony detected in the image of this time (S64).

Specifically, with respect to the images of this time in the time-series, the analyzing apparatus 10 classifies, as the existing colony, the polygonal shape in the captured image of this time, which is most approximate to the contour (polygonal shape) of each of the already-extracted colonies in the images of the last time. The following method for the classification into the existing colony, may be exemplified.

The method involves:

(1) selecting the edge most approximate to the edge of the polygonal shape of the image of the last time.

The method further involves:

(2) selecting, as the existing colony, the polygonal shape in the image of this time, which has the edge most approximate to the centroid of the polygonal shape of the image of the last time.

The method still further involves:

(3) selecting, as the existing colony, the polygonal shape in the image of this time, which has the centroid most approximate to the centroid of the polygonal shape of the image of the last time.

The method yet further involves:

(4) providing points in the sequence from the highest approximation to the polygonal shapes of (1) through (3), and selecting the polygonal shape in the image of this time, which has the highest total point.

The method additionally involves:

(5) selecting, preferentially as the existing colony, when the polygonal shape in the image of this time internally embraces the polygonal shape in the image of the last time with respect to the polygonal shapes of (1) through (4), the polygonal shape of the last time embraced in the polygonal shape in the image of this time. The process in S64 may be said to be a process of detecting existences of the colonies that already occurred in the time-series. This process may be said to detect the existence of the colony that already occurred, based on at least one of borderlines of the shadow areas existing respectively in the plurality of first images captured with the blue light, and the centroid of the area surrounded by the borderlines.

The analyzing apparatus 10 classifies, as the new colonies, the colonies other than the existing colonies. Whereas when the occurrence of the colony is not yet detected at the stage up to the image of the last time (N in S63), the analyzing apparatus 10 detects, as the new colonies, all the polygonal shapes detected in the image of this time (S65). The process in S65 may be said to be a process of detecting the occurrence of the colony of the microorganisms. Accordingly, the processes in FIG. 6 are one example of a process of specifying a shape of the colony, of which the occurrence or the existence is detected. In the first embodiment, the CPU 1, executing the processes in FIG. 6, of the analyzing apparatus 10 is called a first specifying unit. The processes in FIG. 6 are also referred to as a first step of detecting occurrence of the colony of the microorganisms or an existence of the colony occurring already. The processes in FIG. 6 are one example of a process of detecting the occurrence of the colony of the microorganisms or the existence of the colony that already occurred, based on at least one of the borderlines of the shadow areas existing respectively in the plurality of first images captured with the blue light, and the centroid of the area surrounded by the borderlines.

(Processing Example)

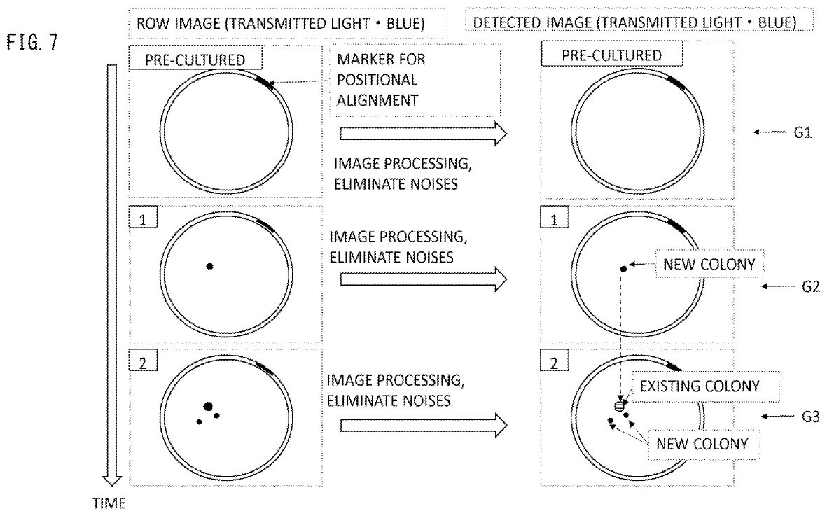

FIG. 7 is a diagram illustrating the time-series images of a transition of how the colony occurs and grows. The illumination is the transmitted light emitted by the blue light LB from under the Schale PD.

In FIG. 7, an arrow extending downward from upward indicates an elapse of the time. The three images arrayed in a left column in FIG. 7 are exemplified as raw captured images of the Schale PD. The three images arrayed in a right column in FIG. 7 are exemplified as processed images obtained by executing the image processing on the raw images and eliminating noises therefrom.

The analyzing apparatus 10 at first captures the images of the Schale PD in the status before entering the culturing step (images in a row G1). In this status, any shadow areas are normally not detected. The shadow areas detected in this status may be determined to be the foreign matters (contaminants). Such being the case, the shadow areas of the foreign matters are eliminated from the subsequent images. This is as described in the process of S28 in FIG. 2.

When the culturing step advances, the new colony occurs (images in a row G2). The shadow area not found out in the images of the last time is determined to be the new colony. Specifically, the analyzing apparatus 10 compares the images of the Schale PD at the stages previously and lately in the time-series of the culturing step, and, when having no coincident positional information of the colonies, determines the shadow area as the new colony. This is as described in the process of S65 in FIG. 6.

The analyzing apparatus 10 compares the images of the Schale PD at the stages previously and lately, and, when the shadow area disappears in the subsequent image (the image of this time), eliminates the shadow area as the foreign matter (contaminant) at that timing. This is as described in the process of S33 in FIG. 3.

When the culturing step further advances, the new colonies additionally occur (images in a row G3). The analyzing apparatus 10 classifies, as the existing colony, the colony existing in the image of this time, which is most approximate to the colony existing in the image of the last time (refer to how the colony is associated with another as indicated by an arrow with a dotted line). The analyzing apparatus 10 classifies, as the new colony, the colony existing in the image of the last time but not classified as the existing colony. In the row G3, the existing colony is illustrated with a circle with hatching, and the new colonies are illustrated with solid black circles.

FIGS. 8 and 9 illustrate a process of positionally aligning the image of the last time with the image of this time. FIG. 8 illustrates a process of aligning central positions of the Schales PD. In FIG. 8, the image of the last time is marked with a letter “previous”. The image of this time is marked with a letter “later”. As in FIGS. 8 and 9, the Schale PD has marks M1, M2 inscribed on the edge portion.

The analyzing apparatus 10, to start with, aligns the central position of the Schale PD in the image of the last time with the central position of the Schale PD in the image of this time. Namely, the analyzing apparatus 10 calculates a difference between a coordinate of the center of the Schale PD in the image of the last time and a coordinate of the center of the Schale PD in the image of this time. The analyzing apparatus 10 relatively moves one image by this difference, thus aligning the positions of the Schales PD.

FIG. 9 illustrates a process of aligning rotation angles of the Schales PD. The analyzing apparatus 10, after aligning the central positions in FIG. 8, aligns the rotation angles by rotating the images. The Schale PD has the marks, M1, M2 attached to the edge portion. The analyzing apparatus 10 calculates angles of the reference positions of the marks M1, M2, e.g., the angles of the central positions of the marks M1, M2 of the image of the last time and the image of this time, and rotates one of the marks.

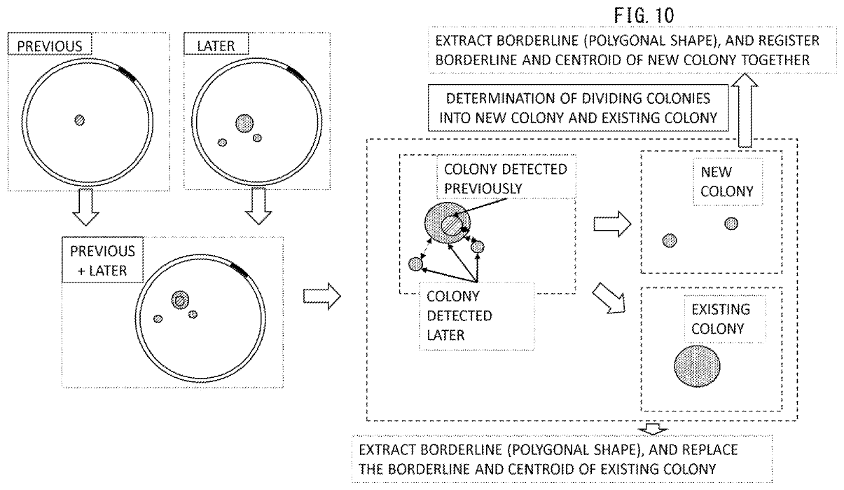

FIG. 10 illustrates a process of classifying the colonies in the image of this time into the existing colony and the new colony. The image marked with the letter “previous” in FIG. 10 takes the polygonal shape representing the colony obtained from the image of the last time in the time-series. The image marked with the letter “later” takes the polygonal shape representing the colony obtained from the image lately in the time-series, i.e., the image of the last time. A symbol “previous+later” represents a status in which both of the polygonal shapes are overlapped with each other.

In this process, the analyzing apparatus 10 calculates a shortest distance of each of the colonies (polygonal shapes) of this time from the colony (polygonal shape) of the last time, and determines the colony, having the short distance to the colony of the last time in the image of this time, as a grown existing colony. In FIG. 10, the colony of the last time is completely embraced by one of the colonies of this time. The colony having the edge most approximate to (the edge of) the colony of the last time is the colony of this time, which embraces the colony of the last time. Such being the case, the analyzing apparatus 10 determines the colony of this time, which embraces the colony of the last time, as the existing colony. The analyzing apparatus 10 executes the processes described so far on all of the colonies of the last time. The analyzing apparatus 10 determines, as the new colony, the colony in the image of this time, which is unable to be associated with any of the colonies of the last time.

The analyzing apparatus 10 registers the borderline (polygonal shape) and the centroid of the new colony. The analyzing apparatus 10 replaces the borderline (polygonal shape) and the centroid of the existing colony with those of the last time, and registers the replaced borderline and centroid.

(Effects of Embodiment)

According to the first embodiment, the analyzing apparatus 10 compares the contours extracted from between the images captured with the blue transmitted light previously and lately in the time-series with the contour extracted from the image captured with the white reflection light. The analyzing apparatus 10 determines the contour of the colony when the two contours are coincident. The contours extracted in the time-series are data acquired by repeating the image processing of the CPU 11. The contours extracted in the time-series are the contours in the images captured in the status of irradiating the transmitted light emitted from the blue light LB suited to the image processing of the CPU 11. This contour is of the colony extracted from the specified colonies that newly occur and the specified existing colonies by comparing the respective images in the time-series with the respective previous images. On the other hand, the contour extracted from the image captured with the white reflection light is easy to be acquired from the image suited to cognition by human eyes. Hence, the analyzing apparatus 10 analyzes the colony accurately by comparing and collating the contour given by processing the image easily visible to the human eyes with the contour extracted in the time-series, and is thereby enabled to provide the operator with the contour of the colony. In that case, the analyzing apparatus 10 analyzes the images in the time-series by processing on one hand, and is therefore enabled to, also when the colonies are overlapped during the culturing step halfway, distinguish between the colonies accurately and count the number of the colonies.

The analyzing apparatus 10, also in the processing in the time-series, classifies, as the existing colony, the polygonal shape in the image of this time, which is most approximate to the contour (or the centroid) of each of the polygonal shapes in the image of this time, and classifies, as the new colony, the colony other than the existing colony. The analyzing apparatus 10 is enabled to determine the occurrence of the new colony by mutually comparing the polygonal shapes obtained from the images in the time-series as such, and accurately distinguishing between the existing colony and the new colony.

The analyzing apparatus 10, when the shadow area existing in the image of the last time in the time-series disappears in the image of this time, determines the disappeared shadow area as the foreign matter, and eliminates the foreign matter from the image of the last time. Accordingly, the analyzing apparatus 10 is enabled to make the analysis by eliminating the foreign matters properly. The analyzing apparatus 10 is also enabled to, owing to this processing, count the number of the colonies accurately.

The analyzing apparatus 10, when it is difficult to compare the image of the last time with the image of this time in the time-series, prompts the operator to input by outputting a message to the display 14. The analyzing apparatus 10 is therefore enabled to obtain a determination of the operator at a proper timing. In that case, the analyzing apparatus 10 is enabled to output, to the display 14, the three-dimensional images captured with the light beams irradiated from the eight directions by the LEDs L1-L8. Consequently, the analyzing apparatus 10 is enabled to assist the operator in determining whether to be the colony or the tarnish of the Schale PD.

The analyzing apparatus 10 causes the fan controller 21 to operate the fan FN to apply the air blow to the Schale PD, thereby cleaning off the foreign matters on the surface of the Schale PD. The analyzing apparatus 10 is therefore enabled to carry out the analysis while suppressing mis-cognition due to the foreign matters as much as possible.

The analyzing apparatus 10 accepts the input from the operator, based on the contour of the colony and the image, and saves a result of how the operator determines an existence or non-existence of the colony. In other words, the analyzing apparatus 10 functions substantially as an apparatus that assists the operator in determining.

The analyzing apparatus 10, when capturing the images with one of the blue light LB and the white light LW at the plurality of stages, captures the images of the Schale PD in a way that changes the light quantity. The analyzing apparatus 10 detects the light quantity in such a stable status that the variation in the number of the counted patterns in each image becomes equal to or smaller than the predetermined threshold value, thus determining the proper light quantity. The analyzing apparatus 10 is therefore enabled to capture the image with the proper light quantity of each of the blue light LB and the white light LW.

The analyzing apparatus 10 displays in the time-series the already-extracted contour of the colony or the already-extracted image being processed underway at each stage of the culturing step. At that time, the operator is able to display, on the display 14, the colony contours at the respective stages or the images being processed underway at the respective stages in a way that freely magnifies and minifies the contours or the images through the manipulation of the manipulator 15. The analyzing apparatus 10 is therefore enabled to properly assists the operator in determining.

### Modified Examples

The first embodiment exemplifies the processes in FIGS. 2 through 4 as the series of processes. It does not, however, mean that the processes of the analyzing apparatus 10 are limited to the processes described above. For instance, an available configuration is that the analyzing apparatus 10 executes the processes in S21 up to S25 in FIG. 2, and another computer connected to a network executes the processes from S26 onward. For example, the image data obtained in the processes of S21 through S25 may be forwarded to another computer, a server and the Cloud storage. Another computer connected to the network refers to the image data saved on the Cloud storage, and may also execute the processes from S26 onward. The analyzing apparatus 10 executes the series of the processes in FIGS. 2 through 4, in which case also the image data and other equivalents may be saved in another computer on the network, the saver on the network, and the Cloud storage. The analyzing apparatus 10 may also execute the processes from S26 onward on the basis of the data acquired in the process of this time and the data acquired in the past and saved in another computer. In that case, the analyzing apparatus 10 may simply download the data acquired in the past from another computer and other equivalents when processing the data acquired in the process of this time.

### Second Embodiment

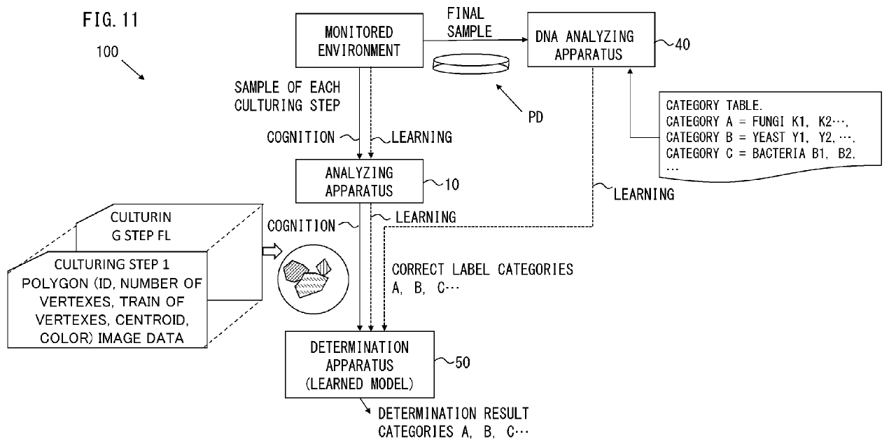

A second embodiment will be described with reference to FIG. 11. FIG. 11 is a diagram illustrating an environment determination system 100 according to a second embodiment. The environment determination system 100 includes the analyzing apparatus 10, a DNA analyzing apparatus 40, and a determination apparatus 50. The analyzing apparatus 10 is the same as the apparatus described in the first embodiment. Specifically, the analyzing apparatus 10, as a unit to output the information representing the state of the colony, inputs, to the determination apparatus 50, items of information exemplified by colony contours, the images and the number of the counted colonies, which are displayed in the processes in S44 through S46 in FIG. 4. The environment determination system 100 may be said to be an analyzing system.

The DNA analyzing apparatus 40 is called a genome analyzing apparatus, a gene analyzing apparatus, or a DNA sequencer. The DNA analyzing apparatus 40 analyzes base sequences of DNAs of the microorganisms within the Schale PD completing the cultivation in the culturing step, and specifies types of the microorganisms. The DNA analyzing apparatus 40 outputs categorized information of the microorganisms, based on the obtained types of the microorganisms. Items of the categorized information are exemplified by “Harmless”, “Low Hazardous Level”, “Intermediate Hazardous Level” and “High Hazardous Level”. The categorized information of the microorganisms may also be roughly categorized such as fungi, yeast and bacteria.

For outputting these items of categorized information, the DNA analyzing apparatus 40 may include a classification table in which to define an associative relationship between a name for specifying the type of the individual microorganism, code identifying information, and the categorized information. The classification table configured as such may be defined corresponding to tasks in the monitored environment. For instance, the monitored environment is exemplified by a biopharmaceutical research and development site, a biopharmaceutical manufacturing site, a food research and development site, and a food manufacturing site, and the classification table may simply be defined corresponding to these sites.

The determination apparatus 50 executes machine learning, based on training data (teacher data) in which classified items of the analysis results by the DNA analyzing apparatus 40 are labeled, and these labels are attached to the analysis results given by the analyzing apparatus 10. The machine learning is what is called deep learning. The determination apparatus 50 includes, as in the case of the analyzing apparatus 10, the CPU, the memory and other equivalent components. The CPU includes the GPU and the DSP as illustrated in FIG. 1. The determination apparatus 50 configures a neural network having a plurality of hierarchical layers among plural virtual nodes, based on the computer programs deployed in the executable manner on the memory. The respective nodes connected to each other via the neural network are disposed in any of the multiple hierarchical layers. However, the neural network may be configured by hardware circuits. For instance, each node includes the CPU, the GPU, the DSP, or a product-sum operation circuit of FPGA, and the nodes are connected via the network. An input layer is a layer, among the multiple hierarchical layers, to which the data are inputted from the analyzing apparatus 10. An output layer is a layer disposed farthest from the input layer among the multiple hierarchical layers.

Each node on each layer among the multiple hierarchical layers receives an output of the node on the layer closer to the input layer, executes a convolution operation exemplified by the product-sum operation assigned to the node itself, or executes a filtering process, and hands over a processing result to the node on the layer closer to the output layer. The output layer receives an output of the node on the layer closer to the input layer, and outputs the classification result. Herein, the product-sum operation or the filtering process attains to an inner product between a coefficient array or coefficients that each node has, and the input data (vector).

At a learning stage, a label of correct data is inputted, together with the input data, from the DNA analyzing apparatus 40. A pair of the input data and the label of the correct data is referred to as the training data (teacher data). A categorized result defined as the output from the output layer is not coincident with the label of the correct data, in which case a difference value between the categorized result and the label of the correct data is propagated backward to the input layer from the output layer. During the backward propagation, the coefficient is adjusted (optimized) in such a direction as to minimize an error in each node on each layer. Such processing at the learning stage is iterated a plural number of times to thereby adjust the coefficient, thus configuring a learned model. Therefore, the learned model has the adjusted coefficients (coefficients of the product-sum operation or the filtering process) that learn features contained in the items of information exemplified by the colony contours, the images and the number of the counted colonies displayed in the processes of S44 through S46 in FIG. 4. In other words, the learned model has the adjusted coefficients based on relationships between the features and the categorized information given by the DNA analyzing apparatus 40 about the features.

At a cognitive stage, a new set of data are inputted to the input layer from the analyzing apparatus 10. The determination apparatus 50 executes a cognitive process on the data set given from the analyzing apparatus 10 by using the coefficients of the respective nodes on the respective layers, which are adjusted at the learning stage. To be specific, the learned model outputs determination results such as categories A, B, C . . . for the input data.

Herein, the input data are, e.g., a set of data obtained in the respective culturing steps. For example, the culturing step is built up by a culturing step 1 through a culturing step FL, in which case the input data are exemplified by the polygonal shapes and the image data obtained in the respective culturing steps. The polygonal shape includes, e.g., ID (Identifying Information), a number of vertexes, a train of vertexes (X and Y coordinate values), coordinates of the centroid, and colors. Note that a coordinate system for defining the vertexes of the polygonal shape is to be set beforehand by the analyzing apparatus 10 and the determination apparatus 50. Incidentally, any one of the data of the polygonal shape and the image data may be sufficient for the input data. Those items of input data are one example of the information representing the state of the colony of the microorganisms.

The input data may include parameters indicating environmental conditions. The parameters are a temperature, humidity, and hours of sunshine. In FIG. 11, the data at the respective stages (the culturing steps 1 through FL) of the culturing step are used, and, however, the processes of the determination apparatus 50 are not limited to such processing. For example, the determination apparatus 50 may input only the data of the cultured result given upon completing the final culturing step FL.

The determination apparatus 50 executes iterative learning on the input data by using the training data attached with the output result of the classification information given by the DNA analyzing apparatus 40, and adjusts the coefficients for the convolutional operation or he filtering process. Accordingly, the determination apparatus 50 performs the machine learning that uses the teacher data including the information representing the state of the colony of the microorganisms, and the labels defined as the categorized results of the microorganisms based on the DNA base sequences acquired by analyzing the microorganisms. The CPU of the determination apparatus 50 is one example of a unit to perform the machine learning.

The learning is performed a predetermined number of times, and the adjustment of the coefficient advances, thereby enabling the determination apparatus 50 to execute the processes at the cognitive stage. To be specific, the determination apparatus 50 outputs the categorized results of the categories A, B, C, . . . for the input data given from the analyzing apparatus 10.

The categorized results are exemplified by “Category A=Harmless”, “Category B=Low Hazardous Level”, “Category C=Intermediate Hazardous Level” and “Category D=High Hazardous Level”. The categorized results may also be exemplified by “Category A=Fungi”, “Category B=Yeast”, and “Category C=Bacteria”. The categorized result may also be subcategorized as exemplified by “Subcategory A=Actinobacteria of True Bacteria Phylum”, and “Subcategory B=High Acidophilic Bacteria of Archaebacteria”. The categorizes result may also be subcategorized as exemplified by “Fungi Subcategories K1, K2, K3, . . . ”, and “Bacteria Subcategories B1, B2, B3, . . . ”. Therefore, the determination apparatus 50 categorize the microorganisms, based on the learned model of the machine learning and on the information representing the state of the newly-acquired colony of the microorganisms. The CPU of the determination apparatus 50 is one example of a unit to categorize the microorganisms and one example of a categorizing apparatus.

As discussed above, the environment determination system 100 according to the second embodiment is enabled to provide the categories of the microorganisms acquired in the monitored environment. Specifically, the environment determination system 100 categorizes the microorganisms, based on the data of the polygonal shape obtained from the colony of the microorganisms, the image data used for the analyzing process, which are defined as the analysis results of the analyzing apparatus 10, or based on those items of data combined with the environment parameters. The categories are not from the strict analysis results in the DNA base sequences by the DNA analyzing apparatus 40 but may be rough categories of the analysis results. The environment determination system 100 provides the rough categories of the sampled microorganisms, whereby it may be enough to enable the determination of whether the strict genome analysis of the sampled microorganisms is required. With the determination as such, the genome analysis is performed by narrowing the microorganisms sampled in the monitored environment down to specific categories. As a result, time and a cost required for the genome analysis are reduced.

### Third Embodiment

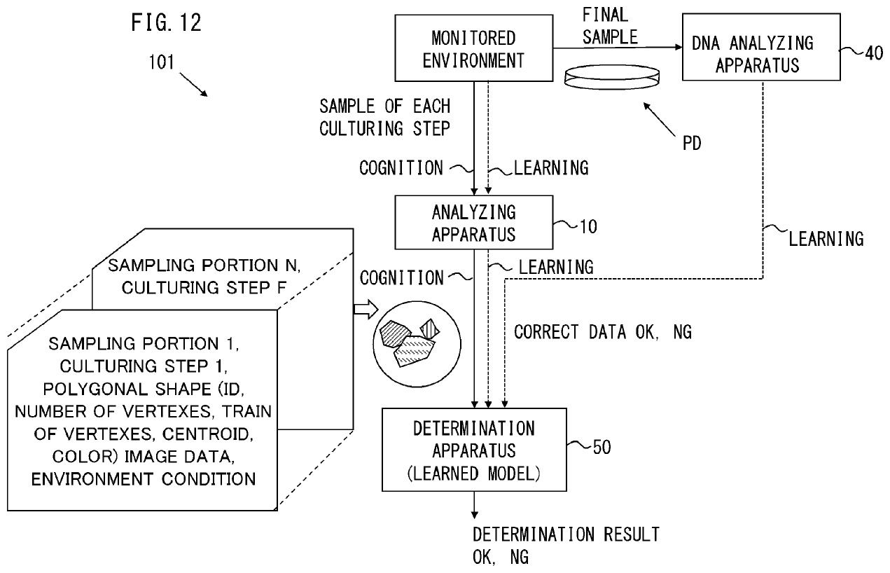

An environment monitoring system 101 according to a third embodiment will hereinafter be described with reference to FIG. 12. The environment determination system 101 may also be said to be the analyzing system. FIG. 12 is a diagram illustrating the environment monitoring system 101 according to the third embodiment. The second embodiment provides the rough category of the microorganisms sampled in the monitored environment, based on the data of the polygonal shape obtained from the colony of the microorganisms, the image data used for the analyzing process, which are defined as the analysis results of the analyzing apparatus 10, or based on those items of data combined with the environment parameters. The third embodiment exemplifies the environment monitoring system 101 that monitors the state of the monitored environment in combination with the environment data representing a condition of the monitored environment together with the analysis results of the analyzing apparatus 10 or the information (hereinafter referred to as analysis data related to the analysis) used for the analysis. Note that the analyzing apparatus 10 inputs, to the determination apparatus 50, the items of information exemplified by the colony contours, the images and the number of the counted colonies displayed in the processes of S44 through S46 in FIG. 4 by way of a unit to output the information representing the state of the colony, similarly to the second embodiment.

The microorganisms are strictly monitored in the site for researching and developing, or manufacturing drugs or foods. Such being the case, the third embodiment will describe the simple and highly-accurate environment monitoring system 101 that uses the analysis data of the analyzing apparatus 10 by properly selecting the environment data representing the condition of the monitored environment.

The environment data representing the condition of the monitored environment is, e.g., data of a sampling portion of sampling the microorganisms. The sampling portion of the sample may be divided into a sampling portion of the airborne bacteria, and a sampling portion of the adherent bacteria. The airborne bacteria are defined as the bacteria that are afloat in the air or fall down. The sampling portion of the airborne bacteria is, when the monitored environment is within a building, exemplified by a position within the building, a room number, a floor, and a position within the room. The position within the room is an in-room position where the Schale PD is placed. The in-room position may also be specified by coordinate values in the coordinate system within the room, and identifying information of a table or a plate on which to place the Schale PD. However, the identifying information of the table or the plate is to be fixed. The sampling portion of the airborne bacteria may also be said to be the position within the monitored environment.

The adherent bacteria are sampled from a sampling target portion exemplified by the human body, e.g., the head and right-and-left arms of the operator working in the monitored environment, or clothing items, e.g., a cap, gloves, front-and-rear surfaces of a sanitary gown, right-and-left sleeves of the sanitary gown, a mask, shoes, doorknobs. Accordingly, these portions are the portions of sampling the adherent bacteria. The parameters indicating the conditions of the monitored environment may include parameters of the environment conditions such as a number of persons, the temperature and the humidity in the room. These items of data are one example of the environment data.

A set of input data is obtained by combining the thus-acquired data, i.e., the position and the sampling portion within the monitored environment, the culturing step of the Schale PD, the data of the polygonal shape (ID, the number of vertexes, the train of the vertexes, the centroid, the color) thereat, the image data, and a quantity or a number of the colonies of the cultured microorganisms. Note that the quantity of the colony is exemplified by a planar dimension of the polygonal shape of the colony. The data of the polygonal shape (ID, the number of vertexes, the train of the vertexes, the centroid, the color), the image data obtained in the time-series during the culturing step, and the quantity or the number of the colonies of the cultured microorganisms, are one example of the analysis data related to the analysis of the analyzing apparatus. In the third embodiment, for instance, the CPU 11 of the analyzing apparatus 10 or the computer (hereinafter referred to as the CPU 11 and other equivalents) cooperating with the analyzing apparatus 10, assumes a case of managing and saving the position and the sampling portion within the monitored environment, and the culturing step of the Schale PD. The CPU 11 and other equivalents create the input data by adding the items of information, i.e., the sampling portion, the culturing step of the Schale PD and the environment conditions to the data of the polygonal shape, the image data, and the quantity or the number of the colonies, which are outputted by the analyzing apparatus 10, and input the created input data to the determination apparatus 50. The position and the sampling portion within the monitored environment, the culturing step of the Schale PD and the environment conditions may be said to be the environment data. Thereat, the determination apparatus 50 acquires the determination result of the DNA analyzing apparatus 40 as a label for the correct data from the DNA analyzing apparatus 40. The determination result of the DNA analyzing apparatus 40 is exemplified by “OK” (no detection of the microorganisms concerned) and “NG” (the concerned microorganisms detected). The determination result is one example of a determination result for the sample of the microorganisms after the sample of the microorganisms is cultured. Note that the CPU 11 of the analyzing apparatus 10 and other equivalents may acquire the determination result of the DNA analyzing apparatus 40 and, after the input data is labeled with the correct data, hand over the labeled input data to the determination apparatus 50.

In the environment monitoring system 101 also, the input data is labeled with the correct data to thereby create the teacher data, and the teacher data is inputted to the determination apparatus 50. The determination apparatus 50 executes the learning process upon the input of the teacher data, and adjusts the coefficient. Accordingly, the determination apparatus 50 performs the machine learning based on the teacher data. The CPU of the determination apparatus 50 is one example of a unit to perform the machine learning.

The learning advances to some extent, and the coefficient is set to a proper value, at which time the determination apparatus 50 executes the cognitive process. To be specific, when receiving the input of the input data including: the environment data exemplified by the sampling portion, the culturing step of the Schale PD and the environment conditions; and the analysis data containing the data of the polygonal shape, the image data and the quantity or the number of the colonies, the determination apparatus 50 outputs the determination result “OK” or “NG”. Therefore, the analysis data of the analysis result by the analyzing apparatus 10 is combined with the environment data indicating the conditions of the monitored environment to create the input data, and the input data enables the environment monitoring system 101 to determine the conditions of the monitored environment. In other words, the determination apparatus 50 may be said to determine the states of the monitored environment, based on the environment data and the analysis data that are newly acquired from the learned model through the machine learning. Accordingly, the CPU of the determination apparatus 50 is one example of a determination unit.

### Modified Examples

According to the second and third embodiments, the analyzing apparatus 10 and the determination apparatus 50 are described as separate apparatuses. The analyzing apparatus 10 and the determination apparatus 50 may be, however, configured as one integrated apparatus. For example, such a configuration may be available that the hardware to attain the learned model is incorporated into the analyzing apparatus 10 and is connected via a bus to the CPU 11. Another available configuration is that the CPU 11 of the analyzing apparatus 10 builds up the neural network on the main storage 12, thereby attaining the learning process and the cognitive process.

<Non-Transitory Computer-Readable Recording Medium>

A program for making a computer exemplified by the IPMC 1, other machines and apparatuses (which will hereinafter be referred to as the computer and other equivalents) attain any one of the functions may be recorded on a non-transitory recording medium readable by the computer and other equivalents. The computer and other equivalents are made to read and execute the program on this non-transitory recording medium, thereby enabling the function thereof to be provided.

Herein, the non-transitory recording medium readable by the computer and other equivalents connotes a recording medium capable of accumulating information exemplified by data and programs electrically, magnetically, optically, mechanically or by chemical action, which are readable from the computer and other equivalents. Among these recording mediums, for example, a flexible disc, a magneto-optic disc, a CD-ROM, a CD-R/W, a DVD, a Blu-ray disc, a DAT, an 8 mm tape, a memory card like a flash memory, are given as those removable from the computer and other equivalents. A hard disc, a ROM (Read-Only Memory) and other equivalents are given as the recording mediums fixed within the computer and other equivalents.

What is claimed is:

1. An analyzing apparatus for a sampling container to sample microorganisms, comprising:
a unit configured to acquire a plurality of first images captured with first visible light in time-series during a culturing step, the plurality of first images acquired by capturing an interior of the sampling container having undergone a process of sampling microorganisms in a monitored environment;
a first specifying unit configured to detect occurrence of a colony of the microorganisms or an existence of the colony having occurred already in the time-series from time-series variations of the plurality of the first images, and to specify a shape of the colony with its occurrence or existence being detected;
a second specifying unit configured to specify the shape of the colony of the microorganisms from a second image acquired by capturing the interior of the sampling container with second visible light; and
a unit configured to display a state of the colony of the microorganisms, based on the shape specified by the first specifying unit and the shape specified by the second specifying unit.

2. The analyzing apparatus according to claim 1, wherein the first specifying unit detects the occurrence of the colony of the microorganisms or the existence of the colony that already occurred, based on at least one of borderlines of shadow areas existing respectively in the plurality of first images, and a centroid of an area surrounded by the borderlines.

3. The analyzing apparatus according to claim 1, wherein the first specifying unit excludes the shadow area existing in the previous image as the shadow area other than the colonies of the microorganisms when the shadow area existing in the previous image does not exist in the later image of the previous and later images in the time-series.

4. The analyzing apparatus according to claim 1, further comprising:
a distinguishing unit configured to distinguish between the shadow area of the colony of the microorganisms and the shadow area other than the colony of the microorganisms; and
a providing unit configured to provide information prompting an intermediary of an operator when the distinguishing unit is difficult to distinguish.

5. The analyzing apparatus according to claim 4, wherein the providing unit displays images captured in a status of illuminating the sampling container with illumination light beams emitted from a plurality of directions.

6. The analyzing apparatus according to claim 1, wherein the sampling container has a transparent bottom, a main body with its upper portion being opened, and a transparent cover portion to cover the upper portion of the main body, and
the analyzing apparatus further includes: an image capturing unit configured to capture the first images and the second images; and
an air blowing unit configured to apply an air blow onto a surface of the sampling container placed in a status of enabling the image capturing unit to capture the images.

7. The analyzing apparatus according to claim 1, further comprising a unit configured to accept confirmation from a user about whether the microorganisms exist, the confirmation being made from the displayed status of the colonies.

8. The analyzing apparatus according to claim 1, further comprising a unit configured to set a light quantity at such a stage that a variation in the number of shadow areas among the plural images captured while changing the light quantity stepwise fulfills a predetermined condition, to a light quantity when capturing the images with one of the first visible light and the second visible light, based on the plurality of images captured while changing the light quantity stepwise by use of the one of the first visible light and the second visible light.

9. The analyzing apparatus according to claim 1, further comprising a unit configured to display states of the colonies in the time-series.

10. The analyzing apparatus according to claim 1, further comprising:
a unit configured to perform machine learning using teacher data containing information representing a state of the colony of the microorganisms, and a label defined as a categorized result of the microorganisms, based on a DNA base sequence obtained by analyzing the microorganisms; and
a unit configured to categorize the microorganisms on the basis of information representing the state of a newly acquired colony of the microorganisms, based on a learned model created through the machine learning.

11. The analyzing apparatus according to claim 1, further comprising:
a unit configured to perform the machine learning using the teacher data including: environment data containing at least one of a position in the monitored environment, in which a sample of the microorganisms is sampled in the sampling container, a sampling target portion, a stage during the culturing step of culturing the sample, and an environment condition in the monitored environment; analysis data related to the analysis by the analyzing apparatus; and a label defined as the determination result of the sample of the microorganisms after the sample of the microorganisms is cultured; and
a unit configured to determine a state of the monitored environment on the basis of newly obtained environment data and the analysis data, based on the learned model created through the machine learning.

12. An analyzing system comprising:
an analyzing apparatus for a sampling container to sample microorganisms; and
a categorizing apparatus,
the analyzing apparatus including:
a unit configured to acquire a plurality of first images, captured with first visible light in time-series during a culturing step, the plurality of first images acquired by capturing an interior of the sampling container having undergone a process of sampling microorganisms in a monitored environment;
a first specifying unit configured to detect occurrence of a colony of the microorganisms or an existence of the colony occurring already in the time-series from time-series variations of the plurality of the first images, and to specify a shape of the colony with its occurrence or existence being detected;
a second specifying unit configured to specify the shape of the colony of the microorganisms from a second image acquired by capturing the interior of the sampling container with second visible light; and
a unit configured to display a state of the colony of the microorganisms, based on the shape specified by the first specifying unit and the shape specified by the second specifying unit,
the categorizing apparatus including:
a unit configured to perform machine learning using teacher data containing information representing the state of the colony of the microorganisms, and a label defined as a categorized result of the microorganisms, based on a DNA base sequence obtained by analyzing the microorganisms; and
a unit configured to categorize the microorganisms on the basis of the information representing the state of a newly acquired colony of the microorganisms, based on a learned model created through the machine learning.

13. An analyzing system comprising:
an analyzing apparatus for a sampling container to sample microorganisms; and
a determination apparatus,
the analyzing apparatus including:
a unit configured to acquire a plurality of first images, captured with first visible light in time-series during a culturing step, the plurality of first images acquired by capturing an interior of the sampling container having undergone a process of sampling microorganisms in a monitored environment;
a first specifying unit configured to detect occurrence of a colony of the microorganisms or an existence of the colony having occurred already in the time-series from time-series variations of the plurality of the first images, and to specify a shape of the colony with its occurrence or existence being detected;
a second specifying unit configured to specify the shape of the colony of the microorganisms from a second image acquired by capturing the interior of the sampling container with second visible light; and
a unit configured to display a state of the colony of the microorganisms, based on the shape specified by the first specifying unit and the shape specified by the second specifying unit,
the determination apparatus including:
a unit configured to perform the machine learning using the teacher data including: environment data containing at least one of a position in the monitored environment, in which a sample of the microorganisms is sampled in the sampling container, a sampling target portion, a stage during the culturing step of culturing the sample, and an environment condition in the monitored environment; analysis data related to the analysis by the analyzing apparatus; and a label defined as the determination result of the sample of the microorganisms after the sample of the microorganisms is cultured; and
a unit configured to determine a state of the monitored environment on the basis of the newly obtained environment data and the analysis data, based on the learned model created through the machine learning.

14. An analyzing method by which a computer analyzes a sampling container to sample microorganisms, comprising:
acquiring a plurality of first images, captured with first visible light in time-series during a culturing step, the plurality of first images acquired by capturing an interior of the sampling container having undergone a process of sampling microorganisms in a monitored environment;
detecting occurrence of a colony of the microorganisms or an existence of the colony having occurred already in the time-series from time-series variations of the plurality of the first images, and specifying a shape of the colony with its occurrence or existence being detected;
specifying the shape of the colony of the microorganisms from a second image acquired by capturing the interior of the sampling container with second visible light; and
displaying a state of the colony of the microorganisms, based on the specified shape of the colony with its occurrence or existence being detected and the specified shape specified from the second image.

15. A computer-readable non-transitory storage medium storing a program of making a computer analyze a sampling container to sample microorganisms, the program comprising:
acquiring a plurality of first images, captured with first visible light in time-series during a culturing step, the plurality of first images acquired by capturing an interior of the sampling container having undergone a process of sampling microorganisms in a monitored environment;
detecting occurrence of a colony of the microorganisms or an existence of the colony having occurred already in the time-series from time-series variations of the plurality of the first images, and specifying a shape of the colony with its occurrence or existence being detected;
specifying the shape of the colony of the microorganisms from a second image acquired by capturing the interior of the sampling container with second visible light; and
displaying a state of the colony of the microorganisms, based on the specified shape of the colony with its occurrence or existence being detected and the specified shape specified from the second image.
